(19) United States
(12) Patent Application Publication
(10) Pub. No.: US 2007/0093744 A1
(43) Pub. Date: Apr. 26, 2007

(54) Permanent thrombus filtering stent

(75) Inventor: David R. Elmaleh, Newton, MA (US)

Correspondence Address: NIXON PEABODY, LLP, 401 9TH STREET, NW, SUITE 900, WASHINGTON, DC, 20004-2128, US

(73) Assignee: Massachusetts General Hospital, Charlestown, MA (US)

(21) Appl. No.: 11/489,726

(22) Filed: Jul. 20, 2006

Related U.S. Application Data
(63) Continuation of application No. PCT/US05/01439, filed on Jan. 19, 2005.
(60) Provisional application No. 60/536,995, filed on Jan. 20, 2004.

(51) Int. Cl. A61B 17/20

(52) U.S. Cl. 604/22; 606/200; 623/1.15

(57) ABSTRACT

A permanent thrombus and plaque filtering stent blocks and/or filters potential emboli in patients undergoing intravascular treatment and/or stent implantation. The stent has a plurality of movable magnetic or ultrasonic agitating elements attached thereto, which when remotely activated move, vibrate or rotate to break up the thrombus, plaque or tissue debris.

### BACKGROUND OF THE INVENTION

1. Technical Field

The present invention relates to a permanent thrombus and plaque filtering stent for blocking and/or filtering potential emboli in patients undergoing intravascular treatment and/or stent implantation, and more particularly to a stent having a plurality of movable magnetic or ultrasonic agitating elements attached thereto which when remotely activated move, vibrate or rotate to break up the thrombus, plaque or tissue debris.

2. Background Art

Coronary artery disease is the leading cause of permanent disability and death in the United States. The coronary arteries supply the blood to the heart muscle. The accumulations of fatty tissue, calcium and cholesterol accumulate in the arteries and limit the flow of the blood supply to the heart. Thus, the major contributors to these types of disease are the gradual plaque formation and substantial closure of the heart arteries (stenosis), as well as, vulnerable plaque formation that is not necessarily associated with narrowing of the artery, but with an inflammatory process that results with gradual thin cap development and cause the artery to be prone to rupture, clot formation and artery blockage.

To treat this blockage percutaneous transluminal coronary angioplasty (PTCA) is used. These procedures open the arteries and allow normal levels of blood flow to the heart muscle to resume. However, this procedure does not eliminate the regrowth and reblockage (restenosis) of the arteries.

Today most if not all angioplasty procedures are associated with the deployment of a metal stent and/or a drug eluting stent that enable the scaffolding of the artery and limit the restenosis. Angioplasty and stent placement cause tissue trauma. The inflation of a balloon in the atherosclerotic plaque area of the coronary artery compresses the plaque, and widens the artery opening. This procedure is ruff and traumatizes the artery wall.

Metal stents are the major device breakthrough in controlling re-narrowing of arteries following angioplasty. Stents comprise latticed or wire mesh metal tubes typically 10-20 mm in length and are used to open blood vessels and prevent buildup of fatty tissue that block the artery, and improve blood flow to the heart. These permanently placed stents reduce the post-angioplasty narrowing, lower chest pain, lower additional surgery, disability and death. Therefore, today the majority of angioplasty procedures are followed by stent placement.

Post angioplasty re-narrowing of a coronary artery occurs in 40 to 50% of patients within 3 to 6 months after the procedure. Stent placement reduces this re-narrowing incidence to 20-30%. To further overcome this problem stents are often coated with drugs that prevent plaque formation to reduce artery reblocking and thus, reduce potential heart attacks, complications and death. With these drug coated stents re-narrowing can be reduced, for example, by to 7 to 10%.

Two major processes cause complications and re-narrowing of the artery following angioplasty and stenting. The first is thrombosis (blood clotting), it occurs mainly during and after the angioplasty. The blood clotting mechanism is a defense reaction of the blood to protect blood leakage from the smashed vessel wall. The use of anti-coagulant drugs during and after the angioplasty reduce this restenosis process to blood clots. In the same manner IIb/IIIa inhibitors (abciximab and eptifabatide) as anti-platelet drugs and/or aspirin and /or ticlopidine or Heparin and Warfarin, a derivative of Coumarin and the like eliminate this problem.

The second process, restenosis, occurs in the form of tissue growth as a slow process of endothelial cell proliferation on the lining of the blood vessel. This form of restenosis is part of the tissue healing and regeneration process and redevelopment arteriosclerosis following the trauma of angioplasty. This plaque formation process happens 3 to 6 months following the procedure. The arteriosclerosis (fat buildup) or new plaque formation is a much slower process and its contribution to blockage during this period is low. In cases where stent restenosis occurs again and again, angioplasty followed by other treatments such as brachytherapy (intra coronary radiation), anti-anginal drug therapy, coronary artery bypass surgery, or Enhanced External Counter pulsation are considered.

As discussed above, a more recent approach to avoid vessel trauma complications resulting from the angioplasty and stenting, is the use of drug eluting stents (DES). To prevent the onset of restenosis the stents are coated with molecules that inhibit the restenosis due to tissue growth. Anti-inflammatory, anti-infective, antibiotic, anti-mitotic, anti-proliferative, and/or anticoagulants are used. Examples of such are Rapamycin, heparin, paclitaxel-eluting, and actinomycin D-eluting stents. These advances will significantly reduce the restenosis rates. A recent clinical study reported on a drug-coated stents that reduced target vessel failure from 21% to 8.6%.

Many medical conditions can lead to emboli formation. Many deaths and incapacitating occurrences could be prevented if large thrombi are prevented from moving from the vascular bed into the heart, brain or lung. An additional closely related disease to coronary artery disease in its formation is carotid artery disease. Carotid artery disease is the result of the narrowing and blockage of the neck arteries which lowers the supply of oxygen-rich blood to the brain. Carotid artery disease is a major cause of stroke. The main treatment for removing plaque from the inner lining of the carotid arteries is carotid endarterectomy. Carotid artery disease is caused by the same factors that contribute to coronary artery arteriosclerosis heart disease, but tends to develop later in life.

As set forth in A Survey of Stent Designs, Min. Invas. Ther. & Allied Technol. 2002: 11(4) 137-147, there are over one hundred stent designs currently being tested or marketed worldwide. The stents vary in material, for example, Ni/Ti, cobalt alloys, stainless, tantalum, etc. Available stents also have a variety of shapes, wire, ribbon, tubing sheets, etc., and geometrical designs, such as rings, helix, open-cell, closed-cell, and hybrid. The stents are made by a variety of fabrication methods, for example, laser, EDM, photochemical etching, barding, etc. Moreover, numerous accompaniments can be provided, such as, radiopaque coating and markers, and drug-eluting coatings. FIGS. 1-13 illustrate some of these prior art stent designs. However, all the stent designs disclosed are open-ended which do not enable filtering.

It can be appreciated that it is imperative to lower patient risk by blocking and/or breaking apart threatening emboli and filtering the broken pieces. As disclosed in U.S. Pat. No. 5,053,008 it is known to use ultrasonic probes to break up an embolus, the broken apart pieces being removed. The patent disclose a multisheathed intra cardiac catheter (not a stent) that could be used to collect and/or break blood clots by a lysing drug or using ultrasound. The clot is trapped in the umbrella (by controlling the opening and closing the umbrella), broken and suctioned after it is drawn into the lumen of the catheter. The main use of this catheter is for introduction into the pulmonary artery to trap vein-forming clots. After the removal of the thrombus the catheter is removed. However, a disadvantage with this device is that it is not permanent and will not prevent future blockage. Furthermore, the mere size of the device would discourage use in treatment of the coronary and carotid arteries.

U.S. Patent Application Publication No. 2003/0153943 discloses disintegrating an obstruction with ultrasonic energy. This patent application discloses a series of potential filter that could be used in vascular surgery. The filters are reinforced membrane filters. However, these filters are not designed to be permanent due to the nature of the membrane.

Thus there is a need for a permanent stent design which can aid in the prevention of emboli from moving downstream and the break-up of emboli and/or thrombi and prevent plaque build-up thereon.

### SUMMARY OF THE INVENTION

It is an object of the invention to provide a closed-end, permanent thrombosis filtering stent, which prevents and/or slows down the incident of vascular reblockage.

Another object of the prevent invention is to provide a permanent filtering stent made of a material and design which allows for a closed end on the stent.

Still another object of the invention is to provide a permanent stent having means for remotely moving portions of the stent to aid in break-up of the thrombosis.

Yet another object of the present invention is to provide additional means insertable within the closed-end, filtering stent to further fracture the thrombosis and remove the particles from the stent site.

In accomplishing these and other objects of the present invention, there is provided a permanent, thrombus filtering stent having a tubular length of flexible mesh having opposed ends. A plurality of movable elements are disposed along the length of the mesh. The plurality of elements are movable to break up surrounding plaque, thrombus or tissue debris.

The plurality of elements further prevent cell aggregation and/or other plaque and thrombus aggregation and initiate the break up of surrounding plaque, thrombus or tissue debris. The elements are remotely activated and controlled to move, vibrate or rotate temporarily in a fashion that prevents plaque and thrombus from forming post balloon and/or catheterization stenting. Moreover, in cases where the formation of plaque and/or thrombi cannot be avoided the permanent stent of the present invention prevents the blockage from moving downstream and allows the use of an ultrasonic tip in combination with the stent to breakup the blockage.

The present invention also comprises an ultrasonic catheter having a tip having a plurality of inlets for blood flow therethrough. An ultrasonic head is located at the tip for breaking apart a thrombus, plaque or tissue debris. A flow initiating device located downstream of the ultrasonic head filters the blood flow and debris through the plurality of inlets and returns the filtered blood.

The present invention further contemplates a method of disintegrating a thrombus, plaque or tissue debris comprising the steps of inserting a self-expandable stent into a vessel having a thrombus, plaque or tissue debris, and activating a plurality of elements movably disposed along a length of the stent to prevent the formation of and/or break apart the thrombus plaque or tissue debris.

The stent of the present invention is a permanent stent that increases the prevention of restenosis and opens the window for extension of stent therapy for various coronary diseases and other vessel therapy intervention.

The permanent stent of the present invention also uses a filtering system to collect potential forming clots due to the stenting procedure. The permanent stent can collect potential clots and debris during the procedure and/or prevent restenosis and trapping of potentially released ruptured plaque and thrombus.

The permanent stent of the present invention also uses a filtering system to collect potential forming emboli in patients prone to stoke, i.e., patients who are aging, suffering from high blood pressure due to hardening of the carotid artery, and those having a history of small stoke occurrences. The permanent stent can collect potential clots and debris during the procedure and/or prevent stoke by trapping the released ruptured plaque and/or thrombus.

The permanent stent of the present invention can tremendously lower the incidence of restenosis, in patients undergoing angioplasty/stent therapy. Furthermore, patients with a high risk of clot formation and potential plaque rapture in the coronary artery, which can cause chest pain, heart attack and massive myocardial infarction and death, or in cases of high risk carotid artery plaque formation and clots that can cause stroke, brain damage or death, or with routine vascular stent graft intervention, will benefit from the present invention. The prevention of these diseases could be improved by lowering tissue and cell aggregation, clots and plaque formation using a remote treatment following the procedure. In extreme cases where clots or plaque are trapped in the closed end of the stent further treatment by the ultrasonic tip to break and remove the debris can be accomplished.

The stent of the present invention can be permanently introduced into the pulmonary artery, for example, in patients with recurring vein thrombosis or with a high risk for pulmonary emboli. One advantage being the prevention of clots that are formed during a vein surgery procedure. Also, the prevention of clots in high-risk patients from future blockage of the lung.

Another advantage of the permanent stent of the present invention is that the closed end thereof lowers the stent edge complications at least one end of the stent.

Still another advantage of the permanent stent of the present invention is its use in coronary sinus stenting where it is necessary to decrease blood flow out of the coronary sinus. The closed end of the stent of the present invention can be easily manipulated to restrict flow as needed by controlling the number and size of the opening(s) in the closed end, in addition to its filtering function.

These and other objects, features, aspects, and advantages of the present invention will become more apparent from the following detailed description of the preferred embodiment relative to the accompanied drawings, in which:

### BRIEF DESCRIPTION OF THE DRAWINGS

FIG. 1 is a perspective view of a known open-ended stent of knitted wire.

FIG. 2 is a perspective view of a known open-ended stent having a backbone and integral markers.

FIG. 3 is a perspective view of a prior art open-ended wire stent welded to form a closed-cell structure.

FIG. 4 is a perspective view of a known open-ended braided stent.

FIG. 5A is a perspective view of a known frame work for an open-ended stent. FIG. 5B illustrates the framework of FIG. 5A covered with a ePTFE material.

FIG. 6 is a perspective view of a known open-ended knitted stent having gold markers.

FIG. 7 is a perspective view of a known balloon-expandable, open-celled stent having peak-to-peak non-flex connections.

FIG. 8 is a perspective view of a prior art open-ended stent each half of which represents a closed-cell tube structure.

FIG. 9 is a perspective view of a known balloon-expandable, open-celled stent having peak-to-valley connections.

FIG. 10 is a perspective view of a known balloon-expandable, open-celled stent having a sequential ring design.

FIG. 11 is a perspective view of a known ratcheting stent design.

FIG. 12 is a perspective view of a known open-ended closed-cell stent.

FIG. 13 is a perspective view of a known self-expanding open-celled, sequential ring design stent.

FIG. 14 is a perspective view of a closed end, permanent thrombosis filtering stent according to the present invention.

FIG. 15 is a perspective view of a remote, external device used to manipulate the ultrasonic and/or magnetic fingers of the stent according to the present invention.

FIG. 16A is a perspective view of the closed end, permanent thrombosis filtering stent according to the present invention having an opening through which the catheter can extend. FIGS. 16B and 16C illustrate the openings in the closed end of the catheter.

FIG. 17A is an enlarged view of the catheter of the present invention and the movable agitating elements thereof. FIGS. 17B and 17C are expanded views of the agitating elements of the present invention.

FIGS. 18A and 18B are a perspective view and top view of another embodiment of a closed end, permanent filtering stent according to the present invention.

FIGS. 19A-19C are perspective views of an ultrasonic device, which is insertable into the stent of the present invention to disintegrate and remove plaque and/or thrombi.

FIG. 20 is a perspective view of the device of FIGS. 19A-C wherein the cap is positioned on the device.

FIG. 21 is a perspective view of the device of FIGS. 19A-20 located within the vessel/stent.

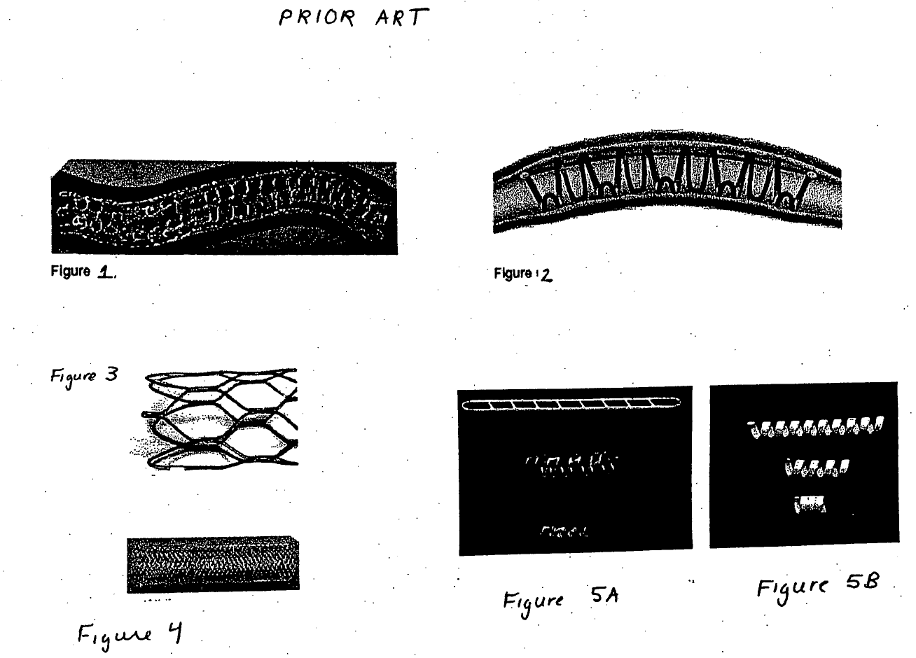

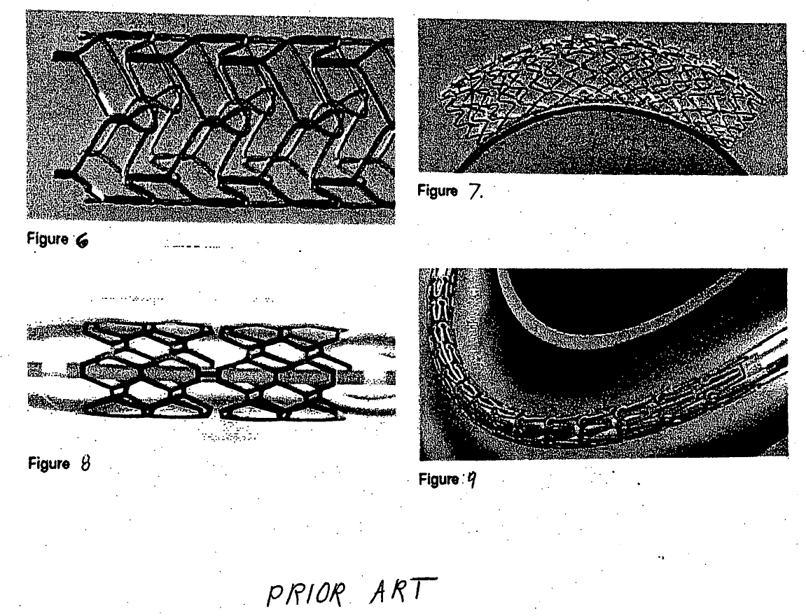

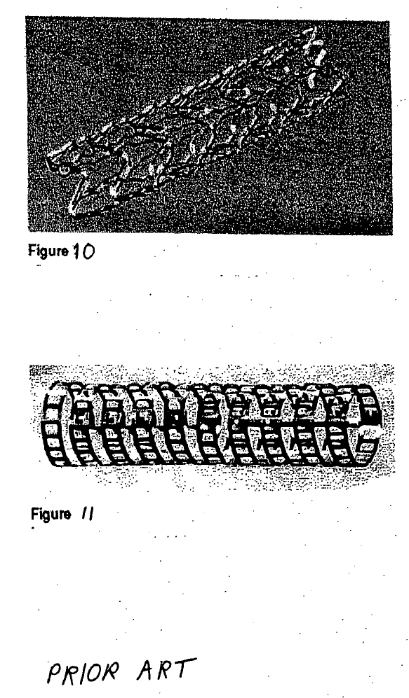

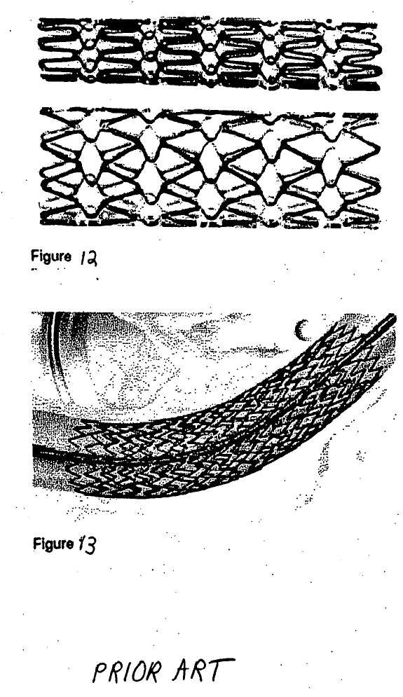

### BEST MODE FOR CARRYING OUT THE INVENTION

For a general understanding of the features of the present invention, reference is made to the drawings, wherein like reference numerals have been used throughout to identify identical or similar elements. FIG. 14 is a perspective view of a stent 20 according to the present invention. Stent 20 is comprised of a length 12 of expandable mesh. It should be appreciated that the mesh can be made from a plurality of materials. The preferred materials have combined properties such as the ones approved by the FDA and which are corrosion resistant, flexible, self-expendable or expendable by a balloon, biocompatible, exhibit strong scaffolding, and which do not produce an effect in MRI. Examples of such materials are 316L stainless steel, palladium/iridium, palladium core/cobalt alloy, and Nickel/titanium. It should be appreciated that the stent of the present invention is not limited to a specific material or combination.

As shown in the embodiment of FIG. 14, the stent is made of self-expanding, open-cell, sequential rings having periodic peak-to-peak non-flexible connections. Once again, it should be appreciated that other stent designs are contemplated by the present invention. Stent 20 has opposed ends 14, 16. End 14 is an open end and will be described further herein. End 16 is a closed end of the stent The closed end can be made of the same mesh as length 12 or can be a mesh of a different material or size attached to the mesh length 12. Preferably, the mesh of closed end 16 should be sized to prevent the passage of cell aggregates, tissue debris and clots that are large enough to result in vessel blockage in the event they should become dislodged causing heart, pulmonary or brain damage. Closed end 16 is also sized to allow for the flow of blood and minute particle.

Preferably, the mesh at closed end 16 should block approximately 20% of blood flow through the artery. More preferably, the mesh should be sized to block a maximum 10%, but the mesh should be sized to block a maximum of 5% or less of the vessel volume. For example, when a 30% closure of an artery occurs blood flow restriction starts to affect the myocardium at rest, but mainly during exercise and stress. Chest pain and possible ischemia may develop. In general, diagnostic imaging at rest and during exercise of one vessel with up to 30% obstruction may not yield definitive positive diagnosis. The flow image may appear normal. A 10% or less will have less on effect on blood flow.

Although closed end 16 is illustrated as being at the top of the stent in the case of stent implementation in the coronary sinus, the stent end 16 would become the bottom as the restriction preferences are in the other direction. This is because the stent design for coronary sinus should allow increased pressure, hence restriction is an improvement.

Closed end 16 could be comprised of any suitable flexible element alloy constituent, for example, stainless steel or palladium core and a cobalt alloy.

Moreover, as discussed above, closed end 16 can be mounted in a fixed or removable manner. Where closed end 16 is a continuation of length 12, it is a continuation of the stent with the gradual closing of the diamond shape openings towards the tissue contact edge of the stent to provide the closed end and shape collapse. A flexible looped ring type of the same material will close the center end of the closed end. The ring is a flexible spring in its expanded or extended form when it is on the catheter.

End 16 is closed after the procedure, however, end 16 could be reopened if it is necessary to perform an additional intervention down stream of the closed end of the vessel. The ring collapses to its closed and normal shape when the catheter is removed. This collapsing of the ring causes the end to close to its final closure. Pushing a catheter through the ring will expand the spring and allow the catheter to move through.

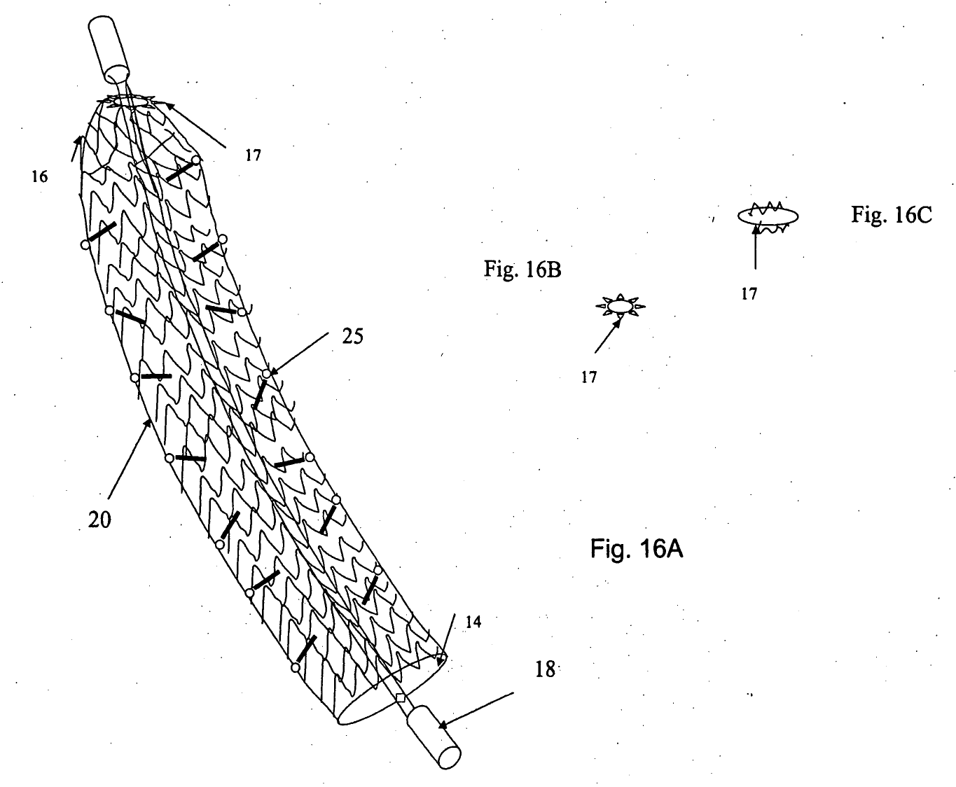

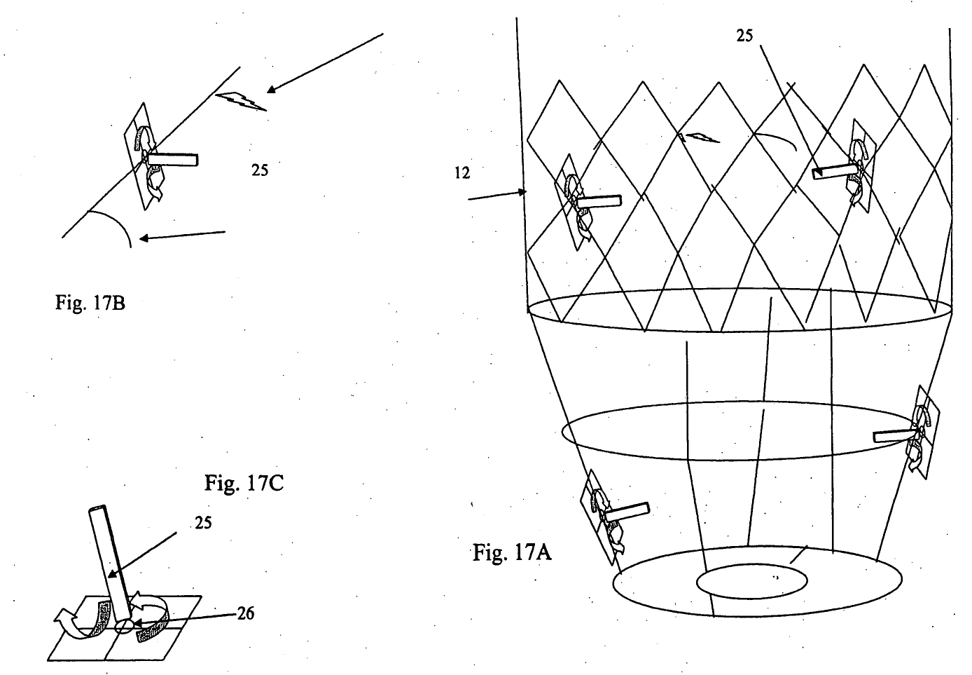

Referring to FIGS. 16A-C, a flexible opening 17 at the top of the meshed closed end will allow reopening of the top. A catheter 18 when pushed through opening 17 will be sufficient to open the top as necessary to move down stream for an additional intravascular intervention. With the catheter lead outside the closed top end it can be pulled so that the self-expanding stent opens and the spring top opening closes to pull the closed end to the center.

Although not shown, closed end 16 can have magnetic or ultrasonic properties. Upon application of an ultrasonic/magnetic force the properties are affected to move the end and enhance disintegration of a thrombus. Moreover, the movement of closed end 16 could be partially limited to a force direction. When ultrasonic force is applied the direction of movement is in all directions. However, when a magnetic force is applied the magnet should move in a direction that will allow, for example, a horizontal move of the closed end section. The vertical movement of the magnetic force is not recommended since it may dislocate the stent body if the force is strong enough.

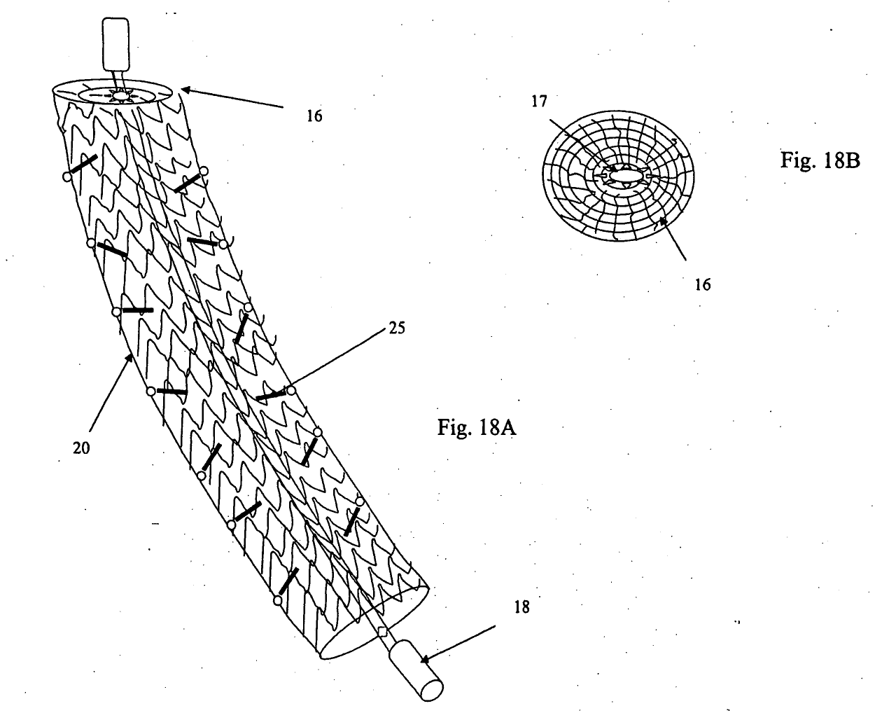

Although the shape of closed end 16 is round, end 16 can have any other shape depending on the particular application. However, the most efficient shapes will be round or elliptic to allow for a large surface area that will minimize edges and flow restriction. As shown in FIGS. 18A and 18B, closed end 16 can be flat having a flexible opening 17. A flat end can be used to control the mesh size and flexibility.

Referring again to FIG. 14, an inflation catheter 18 is provided for deployment of the stent. As shown, the tip of catheter 18 should be disposed at the tip of the stent. It should be appreciated that the stent of the present invention could be a balloon expandable stent, wherein the stent is placed on a balloon and is opened and deployed by the balloon inflation action or a self-expandable stent that will deploy and self expend after the removal of the catheter. In both cases, the stent opens and stays permanently open in its place of deployment to provide permanent scaffolding to the vessel site in treatment.

As shown in FIGS. 14 and 16A, the stent of the present invention includes a plurality of elements 25 disposed along the length of the mesh. Referring to FIGS. 17A-C, elements 25 are small, free moving blade or hair type elements having magnetic/ultrasonic sensitive properties, which are anchored to the stent edges. Elements 25 are movable. attached inside the mesh 12. Elements 25 can be attached to the mesh via rings 26 made of the stent material, as shown in FIG. 17C. Alternatively, elements 25 could be an integral part of the stent. The elements must be flexible to allow for the stent to collapse on the catheter. The rings and elements are allowed to move freely in the stent volume. It should be appreciated that other forms of connection between the elements and the stent are contemplated by the present invention.

Elements 25 could also be flexible bumps disposed on the mesh. The bumps could be of a few microns to a few hundred microns

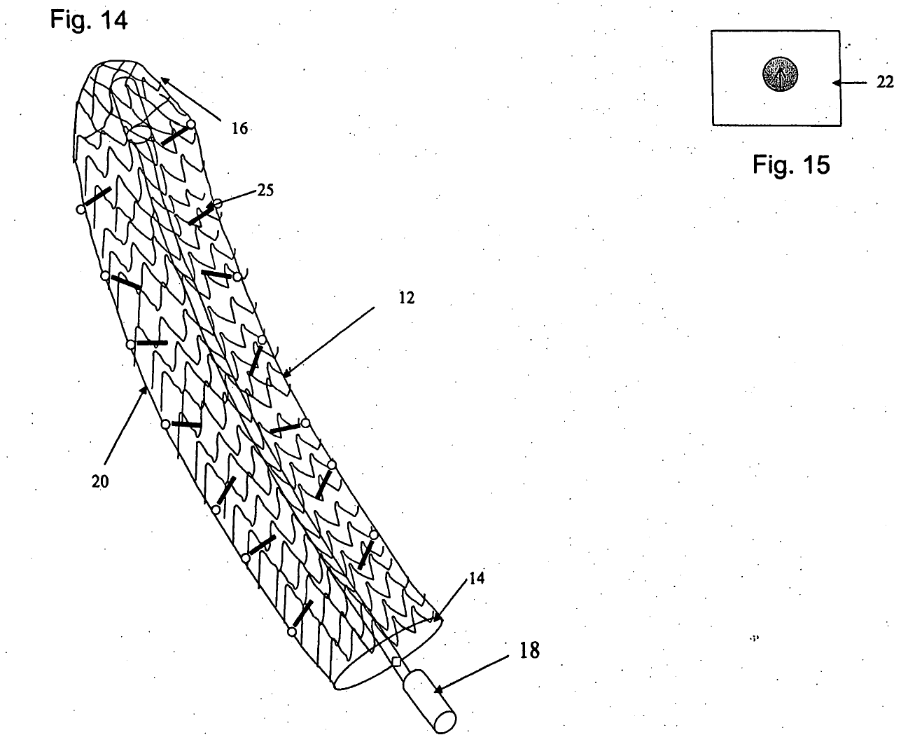

An exterior operated device 22, as shown in FIG. 15, is used to remotely operate elements 15. Device 22 can be a magnetic stirrer or ultrasound vibrator. For example, device 22 can be a handheld portable device operated by batteries or electric power.

The elements are dispersed about the stent length 12 in a statistical plan that allows free flow of blood through the stent. The statistical distribution is to allow for the best mixing possible in the stent volume and to calculate a distribution of elements that does not restrict the blood flow by 20%, more favorably by 10% and more favorably by less than 5%.

The elements 25 can be made of a plurality shapes or materials. For example, as shown in the drawing figures, elements 25 can be wires movably attached to the stent. It should be appreciated that elements 25 can have a diamond, blade, propeller, circular or any other appropriate shape depending on the application. Moreover, the elements can be comprised of a braided wire or plastic hair. With regard to material, any suitable material, which is sensitive to magnetic or ultrasonic energy, can be used. If magnetic energy is applied, magnetic particles or biocompatible Teflon covered magnetic particles. for the remote magnetic agitator 22 can be used to make elements 25. For an ultrasound agitator 22, the stent material is satisfactory.

The size of elements 25 also depends upon the particular stent design and application. For example, the length of the element can range from slightly less than the size of the radius of the expandable stent to less than one tenth of the radius of the expandable stent.

The plurality of elements can be equal-sized or the size can vary along the length of the stent. Moreover, the material of the elements can vary along the length of the stent. Therefore, elements having a higher magnetic property can be placed along the top or middle of the stent to allow for a more vigorous mixing effect in the area of the stent where the emboli or thrombus may be more likely to be located.

The elements and distribution thereof along the stent are designed to block a maximum 20% of the flow of the vessel, and preferably not more than 10% of the vessel volume. Most preferably, elements 25 do not block more than 5% of the vessel volume.

The movement of elements 25 is flexible to move in a desired direction of flow, or against the flow, upon application of magnetic or ultrasonic force. As will be described further herein, elements 25 are flexible to allow the insertion of an ultrasonic tip in the case of a major blockage. Stent 20 and closed end 16 could be coated with anticoagulants or eluting drugs. Moreover, the stent and end can be enclosed within a drug-eluting sleeve. The enlarged surface area added by the closed end and movable elements increase tremendously the amount of the drug attached to the stent and therefore allows for longer or more effective treatment.

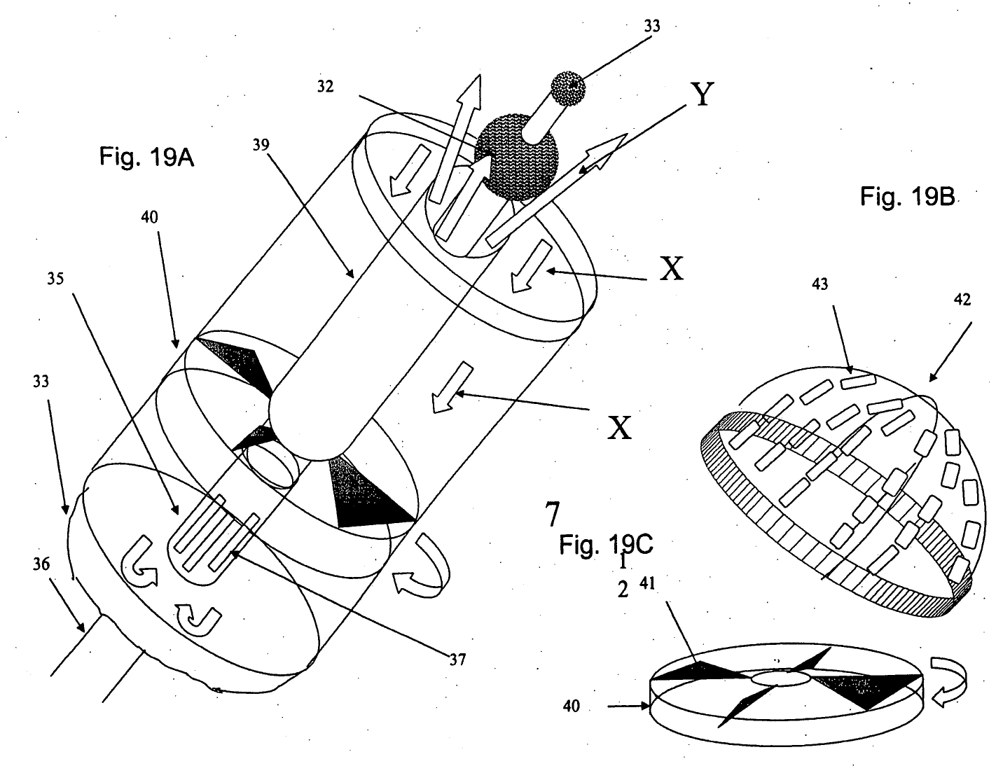

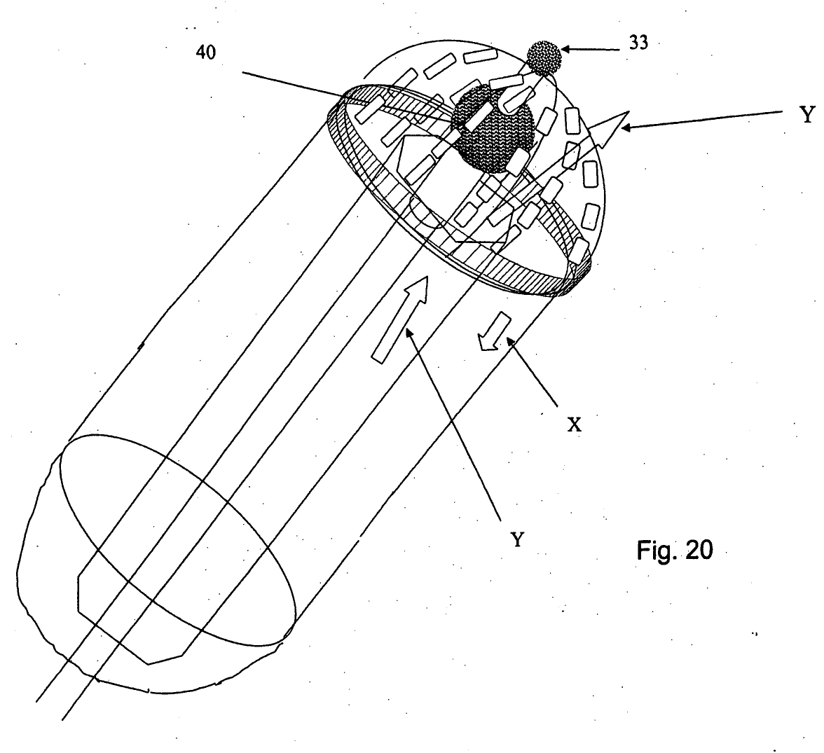

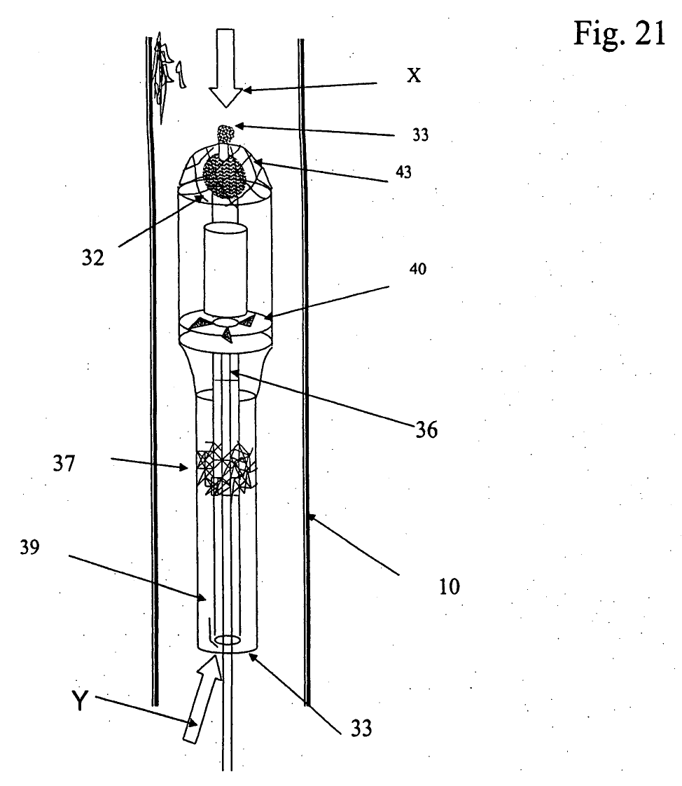

Referring now to FIGS. 19A-21, an ultrasonic catheter 30 is insertable within a vessel 10 and/or stent of the present invention for breaking large thrombus and plaque concentrations disposed in the vessel/stent which were not broken apart by the elements 25. Disposed on an open end 31 of catheter 30 is an ultrasonic device 32 which emits ultrasonic waves to disintegrate the thrombus, plaque or debris tissue that is close to the end of the catheter. Open end 31 acts as an inlet for debris filled blood indicated by arrows X in FIG. 19A and which will be described further herein.

Ultrasonic device 32 can be a brush or other device and includes an optical eye 33 to visualize the area of entry to diagnose the plaque or thrombus and apply various frequencies to break the plaque. The ultrasonic energy is applied to head 32 via an ultrasound catheter cable 36. Cable 36 enters device 40 through a hermetically closed end 33. The bottom closure is necessary to allow for the filtered blood to be sent back into the bloodstream with the blood flow direction. An open bottom would require a flow direction force stronger than the normal blood flow direction.

Catheter 30 also includes a flow initiating device 40 disposed downstream of ultrasonic head 32. Blood flow direction is indicated by arrow X. Device 40 can be a propeller 41 or other device, which draws the blood flow through open end 31. A blood flow return inlet 35 is in fluid communication with flow device 40 and includes a plurality of filters 37 to additionally filter the incoming blood flow allowing more efficient blood return.

Blood entering return inlet 35 is delivered to a filtered blood return tube 39 having an outlet through which the filtered/fractionated blood exits the device, as indicated by arrows Y. A porous cap 42 covers the open end 41 and includes a plurality of filter holes 43 which allow for blood flow through the cap. The device can operate with or without cap 42.

Plaque or tissue debris which has not been broken or only partially broken will undergo an additional mechanical breaking and the left over debris will be trapped by the downstream secondary filter 37.

In operation, the stent 20 of the present invention can be inserted into a blocked or partially blocked vessel and expanded using conventional means. Due to its closed end, the stent can filter potential thrombus formations. Furthermore, due to the plurality of ultrasonic/magnetic elements the thrombus can be disintegrated by application of energy to move, agitate or stir the elements.

One potential use for the stent of the present invention would be for patients undergoing intravascular surgery. The stent of the present invention can also be used in balloon cauterization and stenting. Still other uses would be in vena-cava placement and in patients prone to vulnerable plaque formation. Because of the ultrasonic elements periodic remote treatment can be affected to lower or slow the incidence of vascular re-blockage.

The present invention can also be used for the delivery of gene therapy in hope that introducing genes which can inhibit re-narrowing the arteries and the other that could counteract the response to the vessel injury and therefore limit potential restenosis. Other vascular surgery and endovascular therapy could benefit from the stent design of the present invention. For example, aneurysm repair, atherochotamy, endovascular stent graft, carotid stenting, renovascular surgery, venous surgery, surgical bypass, thrombolysis, vein stripping and other vascular surgery that potentially cause thrombosis.

Although the present invention has been described in relation to particular embodiments thereof, many other variations and modifications and other uses will become apparent to those skilled in the art. It is preferred therefore, that the present invention be limited not by the specific disclosure herein, but only by the appended claims.

What is claimed is:

1. A permanent, thrombus filtering stent for remote or intravascular debris removal, comprising:
a tubular length of flexible mesh having opposed ends, one of said opposed ends being closed to trap thrombus and tissue debris and prevent the same from blocking a blood vessel.

2. The stent of claim 1, further comprising a plurality of movable elements disposed along the length of the mesh, said plurality of elements being movable to prevent surrounding plaque formation.

3. The stent of claim 1, further comprising a plurality of movable elements or bump elevations disposed along the length of the mesh, said plurality of elements being movable to break up surrounding plaque, thrombus or tissue debris or prevent its formation.

4. The stent of claim 2, wherein the plurality of elements are magnetic and capable of being remotely activated.

5. The stent of claim 3, wherein the plurality of elements are ultrasonic and capable of being remotely activated.

6. The stent of claim 4, wherein the plurality of elements are inert to blood and tissue buildup.

7. The stent of claim 5, wherein the plurality of elements are inert to blood and tissue buildup.

8. An ultrasonic catheter comprising:
a tip having a plurality of inlets for blood flow therethrough;
an ultrasonic head at the tip for breaking apart a thrombus, plaque or tissue debris; and
a flow initiating device located downstream of the ultrasonic head for pulling the blood flow and debris upstream through the plurality of inlets and filtering the blood flow and debris.

9. An apparatus for disintegrating and/or preventing thrombus and plaque formation in a blood vessel, comprising:
a filtering stent permanently disposed in the blood vessel, the stent including a tubular length of flexible mesh having opposed ends, and a plurality of movable elements disposed along the length of the mesh, said plurality of elements being movable to break up surrounding plaque, thrombus or tissue debris; and
a device for remotely activating said movable elements to break up a thrombus or prevent a thrombus or plaque from forming.

10. The apparatus of claim 9, wherein one of said opposed ends of said stent is closed to trap thrombus and tissue debris and prevent the same from blocking the blood vessel.

11. The apparatus of claim 9, further comprising an ultrasonic catheter removably insertable within the stent, the catheter including a tip having a plurality of inlets for blood flow therethrough; an ultrasonic head at the tip for breaking apart a thrombus, plaque or tissue debris; and a flow initiating device located downstream of the ultrasonic head for pulling the blood flow and debris upstream through the plurality of inlets and filtering the blood flow and debris.

12. A method of disintegrating a thrombus, plaque or tissue debris and preventing the formation of the same in a blood vessel comprising the steps of:
inserting a self-expandable, permanent stent into a vessel; and
activating a plurality of elements movably disposed along a length of the stent to break apart the thrombus plaque or tissue debris and/or preventing the same from forming.

13. The method of claim 12, wherein the stent has opposed ends, one of said ends being closed and further comprising the step of trapping the disintegrated plaque or tissue debris in the closed end of the stent.

14. The method of claim 12, wherein the step of activating the plurality of elements comprises remotely and temporarily applying an ultrasonic field to activate the elements.

15. The method of claim 12, wherein the step of activating the plurality of elements comprises remotely and temporarily applying a magnetic field to activate the elements.

16. The method of claim 12, further comprising the step of inserting an ultrasonic catheter within the stent and removing, activating the ultrasonic catheter to disintegrate the thrombus, plaque or tissue debris, and filtering the disintegrated thrombus plaque or tissue debris.

17. The method of claim 16, further comprising activating a tip of the ultrasonic catheter is activated to disintegrate the thrombus, plaque or tissue debris and filtering and removing the same with a filtering device disposed within the catheter.
